(19) United States
(12) Patent Application Publication
(10) Pub. No.: US 2014/0314874 A1
(43) Pub. Date: Oct. 23, 2014

(54) Pill for enlarging male genital organ

(71) Applicant: Ronald Chaccour, Zgharta (LB)

(72) Inventor: Ronald Chaccour, Zgharta (LB)

(21) Appl. No.: 13/866,167

(22) Filed: Apr. 19, 2013

(51) Int. Cl. A61K 45/06

(52) U.S. Cl. CPC A61K 45/06
USPC 424/638

(57) ABSTRACT

This pill has the effect of increasing penis size in length and width in male mostly when erected and while flaccid too.

### DETAILED DESCRIPTION OF THE INVENTION

It has been observed that males using multivitamin/multimineral supplement pills especially the ones dedicated for males for 2 days and more, one pill a day experience an increase in penis size, length and width mostly while erected but also if flaccid too.

The increase in the measurements of the penis is about 10% to 15% in length and width.

Multivitamin/multimineral supplement pills usually do not have side effects.

So the use of such pills in order to increase male sexual organ size will not have major side effects.

The best time for taking the pill is 2 to 4 hours before the sexual act.

So a pill weighing 1.53 g and that has the following composition:
Vitamin A 3500 IU
Vitamin C 120 mg
Vitamin D 600 IU
Vitamin E 60 IU
Vitamin K 60 mcg
Thiamin 1.5 mg
Riboflavin 1.7 mg
Niacin 20 mg
Vitamin B6 6mg
Folic acid 300 mcg
Vitamin b12 100 mcg
Biotin 30 mcg
Pantothenic acid 10 mg
Calcium 250 mg
Phosphorus 20 mg
Iodine 150 mcg
Magnesium 50 mg
Zinc 15 mg
Selenium 100 mcg
Copper 0.7 mg
Manganese 4 mg
Chromium 60 mcg
Molybdenum 50 mcg
Chloride 72 mg
Potassium 80 mg
Boron 150 mcg
Nickel 5 mcg
Silicon 2 mcg
Vanadium 10 mcg
Lutein 300 mcg
Lycopene 600 mcg

### Ingredients:

Calcium Carbonate, Potassium Chloride, Ascorbic Acid (Vit. C), Dibasic Phosphate, Magnesium Oxide, dl-Alpha Tocopheryl Acetate (Vit. E), Pregelatinized Corn Starch, Crospovidone. Contains <2% of: Acacia, Ascorbyl Palmitate, Beta-Carotene, BHT, Biotin, Boric Acid, Calcium Pantothenate, Calcium Stearate, Cholecalciferol (Vit. D3), Chromium Picolinate, Citric Acid, Corn Starch, Cupric Sulfate, Cyanocobalamin (Vit. B12), Folic Acid, Gelatin, Hydrogenated Palm Oil, Hypromellose, Lecithin, Lutein, Lycopene, Magnesium Stearate, Manganese Sulfate, Medium-Chain Triglycerides, Microcrystalline Cellulose, Niacinamide, Nickelous Sulfate, Phytonadione (Vit. K), Polyethylene Glycol, Polyvinyl Alcohol, Potassium Iodide, Pyridoxine Hydrochloride (Vit. B6), Riboflavin (Vit. B2), Silicon Dioxide, Sodium Ascorbate, Sodium Benzoate, Sodium Borate, Sodium Citrate, Sodium Metavanadate, Sodium Molybdate, Sodium Selenate, Sorbic Acid, Sucrose, Talc, Thiamine Mononitrate (Vit. B1), Titanium Dioxide, Tocopherols, Vitamin A Acetate (Vit. A), Zinc Oxide.

The intake of one pill containing acetylsalicylic acid with the multivitamin/multimineral supplement pill will increase more the phallus size while erected.

Also the intake of one pill containing flupentixol (as dihydrochloride)+melitracen (as hydrochloride) with one pill containing acetylsalicylic acid with the multivitamin/multimineral supplement pill will increase more the growing effect.

1. The intake of multivitamin/multimineral supplement pills increases penile size in length and width while erected or while flaccid.

2. It enhances physical strength and help keep the erection.

3. Companies producing multivitamin/multimineral supplement pills can sell it as a penis enlarger pill with no proven side effects.
